(19) United States
(12) Patent Application Publication
(10) Pub. No.: US 2019/0053725 A1
(43) Pub. Date: Feb. 21, 2019

(54) NEURAL SIGNAL DATA CAPTURE

(71) Applicant: QUALCOMM Incorporated, San Diego, CA (US)

(72) Inventors: William Henry Von Novak, San Diego, CA (US); Osvaldo Alcala, Chula Vista, CA (US); Stephen Jay Shellhammer, Ramona, CA (US); Daniel Butterfield, Encinitas, CA (US)

(21) Appl. No.: 15/677,650

(22) Filed: Aug. 15, 2017

(51) Int. Cl. A61B 5/04; A61N 1/05; A61B 5/00; G06F 3/01

(52) U.S. Cl. CPC A61B 5/04001; A61N 1/0551; A61B 5/72; G06F 3/015; A61B 5/0031

(57) ABSTRACT

Techniques described herein provide for the detection and capture of neural signals in a manner that compresses data sent from electrodes to controller by several orders of magnitude over current techniques. In particular, embodiments include detecting a threshold amplitude and/or slope of a detected neural signal, and capturing the detected neural signal once these thresholds are met. A threshold timing detector may be implemented as well. Additionally, these thresholds may be configurable to accommodate various factors, such as electrode placement, sensitivity, etc.

### BACKGROUND

### 1. Field

The subject matter disclosed herein relates to neural signal data capture, and more particularly to methods and apparatuses for use in or with a system for detecting neurological activity.

### 2. Information

There are several medical applications that involve sensing and even stimulating neural activity within the brain, such as quadriplegia, Parkinson's, and other medical conditions. This can be done using a neural monitoring system, which use electrodes that can be implanted in a patient's brain to monitor or stimulate neural activity. Current systems may have dozens of electrodes (which may be configured in an array), but future systems may scale to hundreds of thousands of electrodes, or more.

As the number of electrodes in a neural monitoring system increases, the means by which data is retrieved from the electrodes can be complicated by issues related to bandwidth and power. A data stream from the electrodes to a device external to the patient's body, for example, is difficult to send via conventional means, especially at low power levels (important for long battery life.)

### SUMMARY

Techniques described herein provide for the detection and capture of neural signals in a manner that compresses data sent from electrodes to controller by several orders of magnitude over current techniques. In particular, embodiments include detecting a threshold amplitude and/or slope of a detected neural signal, and capturing the detected neural signal once these thresholds are met. A threshold timing detector may be implemented as well. Additionally, these thresholds may be configurable to accommodate various factors, such as electrode placement, sensitivity, etc.

An example electrode of a neural recording system, according to the description, comprises a neural sensor configured to provide an output signal representative of a detected neural signal, and detection circuitry coupled to the neural sensor and configured to determine, from the output signal of the neural sensor, the presence of an action potential in the detected neural signal. The detection circuitry comprises voltage threshold circuitry configured to detect when a voltage of the output signal of the neural sensor reaches a threshold voltage, and slope-detection circuitry configured to detect when a rising slope of the output signal of the neural sensor reaches a threshold slope. The electrode further comprises capture circuitry coupled to the detection circuitry and configured to, based on the determined presence of an action potential, capture a value indicative of an amplitude of the action potential and a value indicative of timing of the action potential, and transmission circuitry coupled to the capture circuitry and configured to send the value indicative of the amplitude of the action potential and the value indicative of the timing of the action potential to a device communicatively coupled with the electrode.

The electrode may comprise one or more of the following features. In some embodiments, the electrode may be implantable. The electrode may comprise circuitry configured to enable adjustment of the threshold voltage, the threshold slope, or both. The slope-detection circuitry may further comprise time threshold circuitry configured to provide an output based on whether the output signal of the neural sensor has met or exceeded the threshold slope for at least a threshold amount of time. The slope-detection circuitry may further comprise circuitry configured to enable adjustment of the threshold amount of time. The capture circuitry may comprise a peak detector coupled with an analog-to-digital converter. The electrode may further comprise a counter configured to provide the value indicative of the timing of the action potential. The counter maybe configured to be reset in response to the detection of the action potential. The slope-detection circuitry may further comprise local potential detection circuitry configured to detect, from the output signal of the neural sensor, a local potential of the detected neural signal, where the transmission circuitry may be further coupled to the local potential detection circuitry and configured to send a value indicative of an amplitude of the detected local potential to the device communicatively coupled with the electrode.

An example method of operating an electrode of a neural recording system, according to the description, may comprise receiving, from a neural sensor, an output signal representative of a detected neural signal, and determining, from the output signal of the neural sensor, the presence of an action potential in the detected neural signal, by determining a voltage of the output signal of the neural sensor reached a threshold voltage, and determining a rising slope of the output signal of the neural sensor reached a threshold slope. The method further comprises, in response to determining presence of an action potential, capturing a value indicative of an amplitude of the action potential and a value indicative of timing of the action potential, and sending the value indicative of the amplitude of the action potential and the value indicative of the timing of the action potential to a device communicatively coupled with the electrode.

The method may comprise one or more of the following features. In some embodiments, the electrode may be implantable. The method may further comprise adjusting the threshold voltage, the threshold slope, or both. The method may further comprise providing an output based on whether the output signal of the neural sensor has met or exceeded the threshold slope for at least a threshold amount of time. The method may further comprise adjusting the threshold amount of time. The method may further comprise operating a counter to provide the value indicative of the timing of the action potential. The method may further comprise resetting the counter in response to the detection of the action potential. The method may further comprise detecting, from the output signal of the neural sensor, a local potential of the detected neural signal, and sending a value indicative of an amplitude of the detected local potential to the device communicatively coupled with the electrode.

An example apparatus, according to the description, comprises means for receiving, from a neural-sensing means, an output signal representative of a detected neural signal, and means for determining, from the output signal of the neural-sensing means, the presence of an action potential in the detected neural signal, by determining a voltage of the output signal of the neural-sensing means reached a threshold voltage, and determining a rising slope of the output signal of the neural-sensing means reached a threshold slope. The apparatus further comprises means for capturing a value indicative of an amplitude of the action potential and a value indicative of timing of the action potential, and means for sending the value indicative of the amplitude of the action potential and the value indicative of the timing of the action potential to a device communicatively coupled with the apparatus.

The apparatus may further comprise one or more of the following features. The apparatus may further comprise means for adjusting the threshold voltage, the threshold slope, or both. The apparatus may further comprise means for providing an output based on whether the output signal of the neural-sensing means has met or exceeded the threshold slope for at least a threshold amount of time. The apparatus may further comprise means for adjusting the threshold amount of time. The apparatus may further comprise means for operating a counter to provide the value indicative of the timing of the action potential. The apparatus may further comprise means for resetting the counter in response to the detection of the action potential. The apparatus may further comprise means for detecting, from the output signal of the neural-sensing means, a local potential of the detected neural signal, and means for sending a value indicative of an amplitude of the detected local potential to the device communicatively coupled with the apparatus.

An example non-transitory computer-readable medium, according to the description, has instructions embedded thereon for operating an electrode of a neural recording system. The instructions comprise computer code for receiving, from a neural sensor, an output signal representative of a detected neural signal, and determining, from the output signal of the neural sensor, the presence of an action potential in the detected neural signal, by determining a voltage of the output signal of the neural sensor reached a threshold voltage, and determining a rising slope of the output signal of the neural sensor reached a threshold slope. The instructions further comprise computer code for capturing a value indicative of an amplitude of the action potential and a value indicative of timing of the action potential, and sending the value indicative of the amplitude of the action potential and the value indicative of the timing of the action potential to a device communicatively coupled with a computing device operating the electrode.

The example non-transitory computer-readable medium can further comprise one or more of the following features. The instructions may further comprise computer code for adjusting the threshold voltage, the threshold slope, or both. The instructions may further comprise computer code for providing an output based on whether the output signal of the neural sensor has met or exceeded the threshold slope for at least a threshold amount of time. The instructions may further comprise computer code for adjusting the threshold amount of time. The instructions may further comprise computer code for operating a counter to provide the value indicative of the timing of the action potential. The instructions may further comprise computer code for resetting the counter in response to the detection of the action potential. The instructions may further comprise computer code for detecting, from the output signal of the neural sensor, a local potential of the detected neural signal, and sending a value indicative of an amplitude of the detected local potential to the device communicatively coupled with the electrode.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is a simplified cross-sectional diagram illustrating a wireless neural monitoring system, according to an embodiment.

FIG. 2 is an illustration of a graph, plotting the amplitude of an action potential of a neuron over time.

FIG. 3 is an example graph of voltage measured by an electrode over time.

FIG. 4 is an example analog circuit of an electrode of the neural monitoring system (e.g., such as electrode of the neural monitoring system illustrated in FIG. 1).

FIG. 5 is a block diagram of a controller that can be utilized with the circuit of FIG. 4 to capture information regarding a detected action potential, according to an embodiment.

FIG. 6 is a timing diagram illustrating the timing of various signals (voltages) in the circuit of FIG. 4, including input and output signals of the controller of FIG. 5.

FIG. 7 is a block diagram of a digital circuit used to detect and capture information regarding action potentials, according to an embodiment.

FIG. 8 is a flow diagram of an example method of capturing neural signals with an implantable electrode, according to an embodiment.

### DETAILED DESCRIPTION

Illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

It will be understood by a person of ordinary skill in the art that, although the embodiments provided herein are directed toward neural monitoring systems utilizing implanted electrodes, which may be implanted in the brain for medical purposes, other applications and embodiments are contemplated.

As previously discussed, neural monitoring systems can use large arrays of electrodes implanted in a patient's brain to monitor neural signals. In analog systems, these signals can be passed via multi-conductor wiring or other means from the electrode array to a device external to patient's body, where the signals are amplified and converted by an A/D converter and read by a computer. The multi-conductor wiring bundles between the implant and external device can be obtrusive, and replacing them would improve the patient's quality of life and reduce health risks. But attempting to digitize information provided from the implanted electrodes to the external device can be very difficult to implement due to bandwidth concerns, especially as the number of implanted electrodes increases. For example, in a system having 10,000 electrodes, if each electrode sends a 20 bit, 10 KHz stream of data, the resulting data stream to the external device would be 2 Gb/s. Such a data stream is difficult to send via traditional wireless means, especially at low power levels, which is important for low power consumption and minimal heat generation (which can be important for neural implants). Furthermore, such systems typically multiplex the input signals, but parasitic capacitance limits how many signals can be multiplexed into one line, and amplifiers used for multiplexing consume power that is in short supply in a brain implant.

To address these and other issues, embodiments described herein utilize techniques for detecting and capturing neural signals in an efficient manner that results in the reduction of data sent from electrodes to controller by several orders of magnitude. In particular, embodiments include detecting a threshold amplitude and/or slope of a detected neural signal, and capturing the detected neural signal once these thresholds are met. A time threshold may be implemented as well. Additionally, these thresholds may be configurable to accommodate various factors, such as electrode placement, sensitivity, etc.

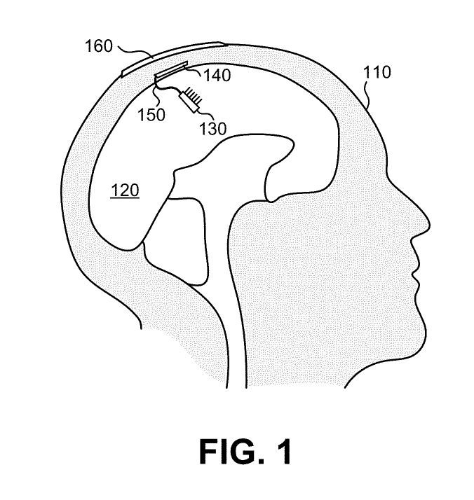

FIG. 1 is a simplified cross-sectional diagram illustrating a wireless neural monitoring system, according to an embodiment. Here, a patient's head 110 is illustrated, indicating a portion the brain 120 and a two-part implant comprising an electrode array 130 and an electronics platform 140, connected via a wired or optical communication link 150. A third component of the system, an external controller 160, is also illustrated. It can be wirelessly connected to the electronics platform 140 (which may be implanted between the brain and skull) and may remain outside the skull, on or near the patient's body. This can provide wireless power and bidirectional radio frequency (RF) communication with the implant. Such wireless communication can employ any of a variety of short-range wireless technologies, including Bluetooth, IEEE 802.11 Wi-Fi, near-field communication (NFC), and/or other wireless technologies. According to some embodiments, data may be communicated in a secure fashion (e.g., using any of a variety of encryption techniques).

It will be understood, however, the configuration and context illustrated in FIG. 1 are provided as an example. A person of ordinary skill in the art will recognize that the techniques described herein can be applied to various types of neural (or even non-neural) implants. For example, some embodiments may combine the electronics platform 140 and external controller 160 into a single physical module, which may be located inside or outside the body. Thus, in some embodiments, the communication link 150 may comprise a wire or optical fiber bundle running from the electrode array 130 directly to a device external to the patient's body. In some embodiments, the electrode array 130 may communicate wirelessly with the electronics platform 140 and/or external controller 160.

The electrode array 130 may comprise various electrodes and/or other elements configured to sense brain activity and optionally cause stimulation of the brain via electrical, optical, and/or other means. In some embodiments, electrodes may not be configured in an electrode array 130, but may instead assume an alternative configuration. One such alternative configuration, for example, may include multiple, individually-configurable electrodes.

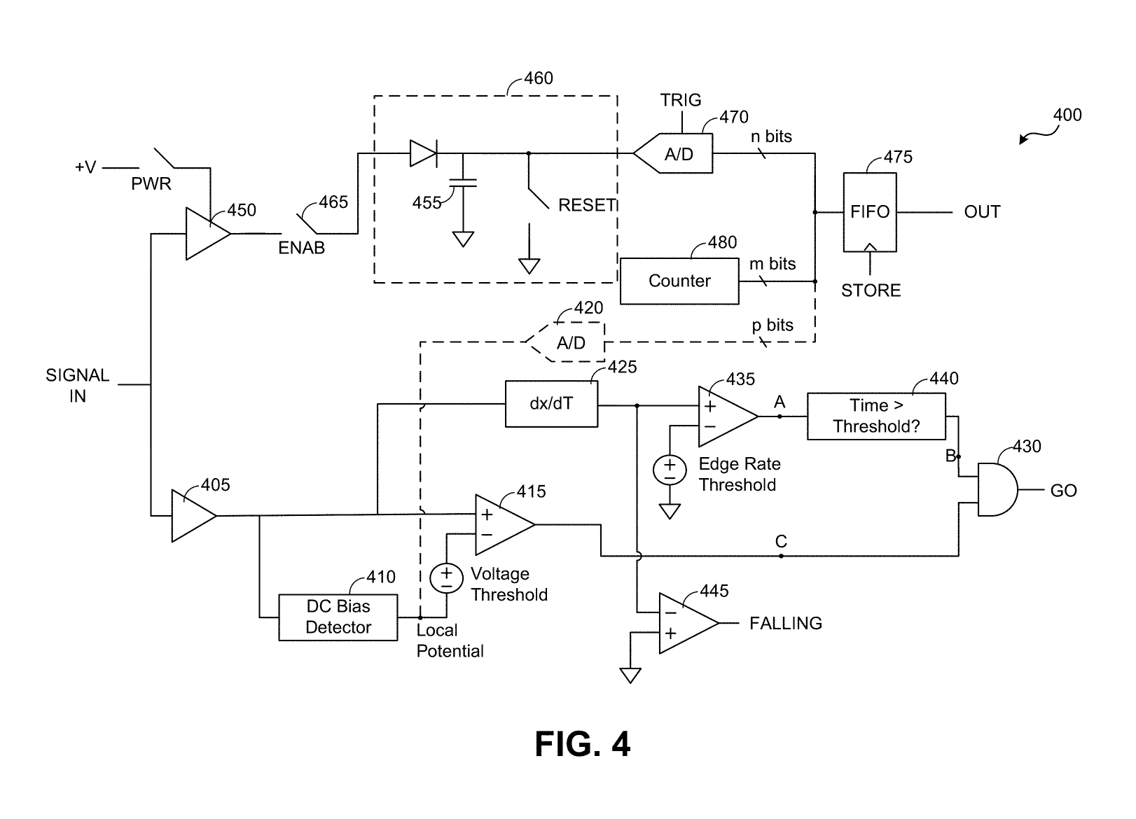

A person of ordinary skill in the art will appreciate the basic hardware components of the electronics platform 140 and/or electrode array 130. This can include, for example, a power source, processing unit, communication bus, volatile and/or non-volatile memory (which may comprise a non-transitory computer-readable medium having computer code for execution by the processing unit), transceiver, antenna, etc. Each electrode in the electrode array 130 may further comprise a sensor (or probe) configured to detect neurological activity, as well as circuitry connected to the sensor for processing the detected neurological activity. Such circuitry is illustrated in FIGS. 4-7 and described in more detail below.

As noted above, the neural monitoring system may comprise hundreds or thousands of electrodes, although alternative embodiments may include a smaller or larger number of electrodes than this. The larger the number of electrodes, the more information regarding neurological activity can be monitored. Specific neurological activity detectable by the electrodes includes “action potentials,” which are electrical signals generated by neurons.

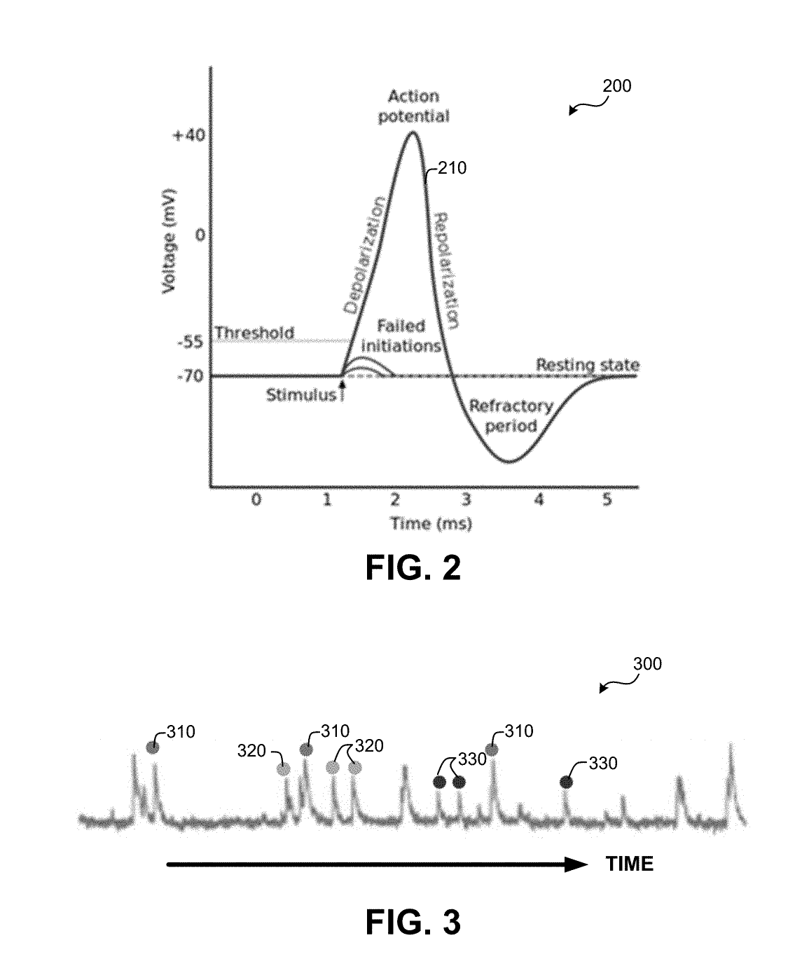

FIG. 2 is an illustration of a graph 200, plotting the amplitude of an example action potential 210 (in millivolts) of a neuron over time (in milliseconds). Here, the local potential during a resting state (e.g., at 0 ms) is −70 mV. At roughly 1 ms, the voltage is increases in response to a stimulus (e.g., from another neuron or other source). If the voltage reaches a threshold level (−55 mV, in this case) an action potential will result, peaking at roughly 40 mV. (Otherwise, if the voltage does not reach the threshold level, it is considered a failed initiation, which can be ignored.) The full action potential 210 includes a depolarization period of rising voltage, a repolarization period after the peak voltage, and a refractory period following the positive spike in voltage. As illustrated on the x-axis of the graph 200, whole process may occur over the course of just a few milliseconds. The distinctive shape and timing of action potentials can help make them relatively easily identifiable to neural monitoring systems.

FIG. 3 is an example graph 300 of voltage measured by an electrode over time. Here, an electrode measures three different series of action potentials, a first, high-voltage series 310, a second, moderate-voltage series 320, and a third, low-voltage series 330. (The shaded dots above the spikes in amplitude (action potentials) are used simply as labels to identify the spikes.) Because neurons (and other nerves) typically have the same depolarization voltage over time, and since sensed potential is attenuated with distance, the different series of action potentials may represent action potentials from three different neurons.

According to techniques described herein, electrodes can include circuitry that may be dynamically adjusted (e.g., after the electrodes have been implanted) to configure how action potentials are identified and reported to a controller device. For instance, a voltage, slope, and/or timing threshold may be used to filter out a series of action potentials in FIG. 3 (e.g., a voltage threshold can be set to filter out the low-voltage series 330). Furthermore, data may be compressed so that data is conveyed only when desired action potentials (those not filtered out) are detected, which can reduce the bandwidth required for conveying neural signal information from the electrodes to the controller device significantly.

In other words, embodiments described herein take advantage of the periodicity and predictability of neural signals to compress these signals by several orders of magnitude. According to embodiments, an electrode may include an analog circuit with a peak detector used to capture the peak of a firing potential. The circuit may further comprise a relatively low-speed analog to digital (A/D) converter to convert the peak value of the signal to a digital sample at (substantially) the same time a timestamp is captured to identify a time the signal was detected. Optionally, the local potential may be sampled so the local potential can be measured. (Local potential may be transmitted less frequently since it changes slowly. The actual potential can later be reconstructed by adding the local potential to the sampled peak.) Thus, for a neural monitoring system, such as the neural monitoring system illustrated in FIG. 1, the reduction in required bandwidth between the electrode array 130 to the electronics platform 140 and between the electronics platform 140 and the external controller 160 can result in a less obtrusive communication link 150 and/or less heat dissipation (and power consumption) due to multiplexing and/or performing other communication-related functions.

FIG. 4 is an example analog circuit 400 of an electrode of the neural monitoring system (e.g., such as electrode 130 of the neural monitoring system illustrated in FIG. 1). Here, the circuit 400 receives a SIGNAL IN input signal from the electrode's sensor (which may detect one or more action potentials from one or more neurons, similar to that illustrated in FIG. 3). The signal is buffered and amplified by low-power amplifier 405 that may be always on. This low-power amplifier 405 (which may have a relatively low fidelity and bandwidth) can enable detection (but not necessarily measurement) of the signal, as described below. The application provided by the low-power amplifier 405 can be important because neural signals are characterized by both small potentials (e.g., 10 μV to 100 mV) and high impedance (e.g., up to 1 megaohm). Thus, the low-power amplifier 405 may have a relatively high input impedance (e.g., well over 1 megaohm) and, in some embodiments, a high amplification (G=1 to 10,000). (That said, A/D converters may operate with millivolts references, which may reduce or eliminate the need for much gain.) It will be understood, however, that the low-power amplifier 405 may not need to pass the signal with significant fidelity, linearity, or bandwidth, as long as the bandwidth is sufficient for detection purposes as described in further detail below.

The low-power amplifier 405 can output to three subsystems.

First, a DC bias detector 410, which recovers a DC bias from the rapidly-changing output of the low-power amplifier 405. It is used to provide a reference for the voltage threshold detector (comparator 415) and a reference for the local potential A/D converter 420. In some embodiments, the DC bias detector 410 may comprise a low-pass filter.

Second, a voltage threshold detector. This comprises a comparator 415 that compares the output of the low-power amplifier 405 to a DC reference voltage generated by the DC bias detector 410. If the voltage input exceeds the DC reference by more than an offset (the voltage labeled “Voltage Threshold,” which may be adjustable), the output, which feeds an input of an AND gate 430, becomes true. Thus, small changes in the voltage of the output of the low-power amplifier 405 can be filtered out. For example, if the DC bias is 10 mV, the voltage threshold may be set to 15 mV, to remove any signal smaller than a 5 mV spike above the local potential.

Third, a differentiator 425. The differentiator provides an output that is the first derivative (with respect to time) of the output of the low-power amplifier 405. This represents the rate of change of the SIGNAL IN (e.g., the “slope” of a detected action potential).

The output of the differentiator 425 is applied to two comparators. The first comparator 435 compares the rate of change to a reference (the voltage labeled “Edge Rate Threshold,” which may be adjustable). If the rate of change exceeds the reference, then the output (node A, the input to timer 440) becomes true. Ensuring that at least the minimum rate of change occurs, can filter out certain increases in local potential that may happen when a lot of nearby neurons are firing, where these increases are relatively rapid in terms of local potential, but not rapid enough to meet the threshold rate of change. This indicates the beginning of spiking amplitude detected in the SIGNAL IN. The timer 440 is activated by the true output of the comparator 435, and, if the output is true for longer than a time threshold (which also may be adjustable), then the output of the timer 440 can go true, indicating the beginning of the spiking amplitude has an appropriate duration, which may be the beginning of an action potential. This helps filter out AC noise and false triggers. In some embodiments, for example, the time threshold may be set as 1 millisecond. (In other embodiments, the time threshold may be longer or shorter.) The output of the timer 440 is then fed to the other input of the and gate 430.

The second comparator 445 looks for the downslope of a detected action potential, outputting a FALLING signal when the downslope is detected, which can trigger certain processes, as detailed below.

At this point, two signals arrive at the AND gate 430. The first is the trigger from the slope detector (differentiator 425, comparator 435, and timer 440). The second is the trigger from the voltage level detector (DC bias detector 410 and comparator 415). If both of these signals are true, then the output of the AND gate goes true. This is the GO signal that begins the process of capturing information regarding a detected action potential. This can be enabled via controller.

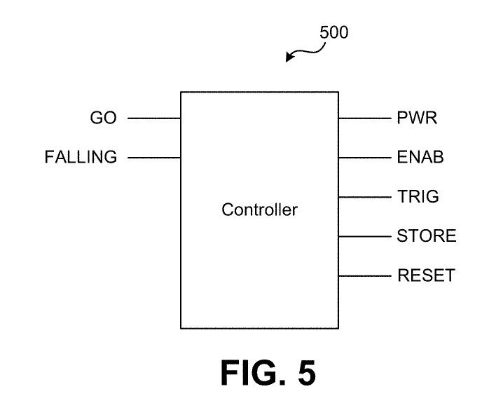

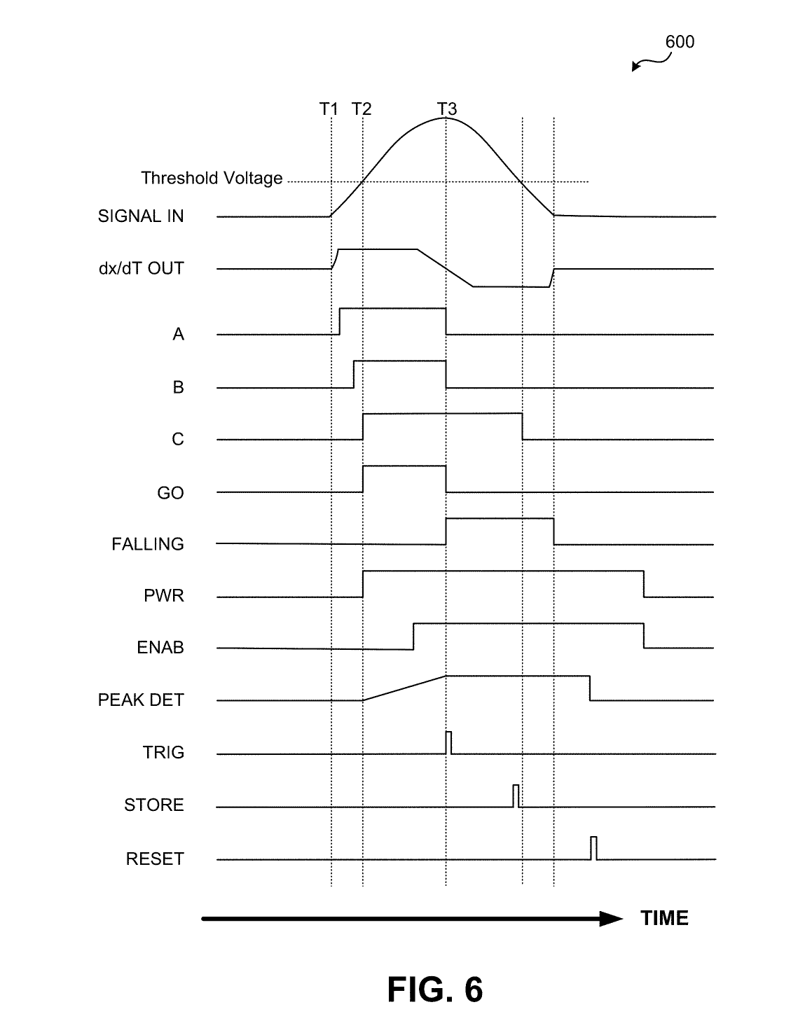

FIG. 5 is a block diagram of a controller 500 that can be utilized with the circuit 400 of FIG. 4 to capture information regarding a detected action potential, according to an embodiment. The controller 500 can be a sequencer that ensures the output signals initiated by the GO signal occur in the correct order and with the correct timing. This functionality may be implemented by a state machine using digital logic, for example, although a person of ordinary skill in the art will appreciate that alternative embodiments additionally or alternatively utilize other circuitry and/or software. As illustrated, the controller 500 receives GO and FALLING input signals, and outputs PWR, ENAB, TRIG, STORE, and RESET signals, as illustrated in the timing diagram of FIG. 6.

FIG. 6 is a timing diagram 600 illustrating the timing of various signals (voltages) in the circuit 400 of FIG. 4, including input and output signals of the controller 500 of FIG. 5. A description of the signals and functionality for capturing information regarding a detected action potential is described below.

First, to discuss the signals of the timing diagram 600 related to detection of the action potential, at time T1 the amplitude of the SIGNAL IN signal begins to rise. In response, the output of the differentiator, dx/dT OUT, provides a signal of the time derivative of the SIGNAL IN signal. The voltage at node A (the output of comparator 435) goes true when dx/dT OUT exceeds the Edge Rate Threshold. When the voltage at node A is true for a threshold of time, as determined by the timer 440, the voltage at node B (the output of the timer 440 and first input to the AND gate 430) goes true. Once the amplitude of the SIGNAL IN signal (as amplified by the low-power amplifier 405) reaches the Voltage Threshold at time T2, the voltage at node C (the output of comparator 415 and second input to the AND gate 430) goes true, and the GO signal (the output of the AND gate 430) is asserted. It will be understood that, depending on the values of the Voltage Threshold and the time threshold, the timing of these signals may vary. For example, the Voltage Threshold may be reached before the time threshold, and thus the voltage at node C may go true before the voltage at node B.

Once the GO signal is asserted, the controller 500 can begin the capture sequence by asserting the PWR signal this turns on the high-power amplifier 450. The high-power amplifier 450 may be configured to have sufficient bandwidth, gain, and output drive to accurately amplify and buffer the SIGNAL IN signal. For example, the high-power amplifier 450 can be configured with sufficient output drive to charge the peak-storage capacitor 455 of the peak detector circuit 460. As such, the high-power amplifier 450 may have a relatively high-power output compared with the low-power amplifier 405. Because the high-power amplifier 450 will be powered only when the GO signal is asserted, the high-power amplifier 450 may not have a large impact on the design's overall power consumption. In some embodiments, the gain of the high-power amplifier 450 and/or the gain of the low-power amplifier 405, may be programmable.

Referring again to the timing diagram 600, after the output of the high power buffer settles, the controller causes the ENAB signal to go true. This closes the switch 465 that connects the high-power amplifier 450 to the peak detector circuit 460, and charges the peak-storage capacitor 455 of the peak detector circuit 460. The peak detector circuit 460 tracks the amplified signal (the output of the high-power amplifier 450) as it rises, then holds the peak voltage of that signal. (The output of the peak detector circuit 460 is indicated by the PEAK DET signal illustrated in the timing diagram 600.)

As illustrated in the timing diagram 600, when the amplitude of the SIGNAL IN signal begins to fall at time T3, dx/dT OUT (the output of the differentiator 425) becomes false, and the comparator 445 asserts the FALLING signal. This causes the controller to assert the TRIG signal (the length of which can be sufficient to activate the A/D converter 470) causing the A/D converter 470 to convert the peak signal (captured by the peak-storage capacitor 455 of the peak detector circuit 460) into a digital value having n bits. It will be appreciated that the A/D converter 470 can be relatively slow and low power, because it receives the output of the peak detector circuit 460. Thus, a high sampling rate may not be required. That said, in alternative embodiments where waveforms are captured (discussed in more detail below) a faster sampling rate may be used.

After providing the A/D converter 470 sufficient time to convert the peak signal, the controller can then assert the STORE signal, which enters a digital word into a first-in, first-out (FIFO) 475 memory buffer, which captures not only the n bits from the A/D converter 470, but also a value from a counter 480 having m bits, providing a time value for when the action potential was detected.

The counter 480 can be implemented in a variety of ways, depending on desired functionality. In some embodiments, for example, the counter 480 may be reset after every detected action potential, so that the value captured in the FIFO 475 represents a length of time since a previous action potential was detected. In other embodiments, the counter 480 may be free running, in which case the value also represents a length of time since a previous action potential was detected (however, this would be determined by the controller device by comparing a current value with a previous value). In either case, the counter 480 may comprise a rolling counter configured to measure length of time between detected action potentials without rolling over. That is, the number of bits of the counter 480 may be selected such that the capacity of the counter is capable of counting the longest foreseeable length of time between two detected action potentials, and the clock rate may be set to a desired temporal resolution.

Optionally, the FIFO 475 may also capture the local voltage potential from the output of the DC bias detector 410, which may be converted to a digital value of p bits by A/D converter 420.

At this point the FIFO 475 has the data, and, according to some embodiments, the data can be withdrawn from the FIFO by a higher level processor as time permits. In some embodiments, there may be a mechanism of semaphores to indicate that the data is available, similar to those used in traditional FIFO designs. Moreover, because a neural monitoring system may contain hundreds or thousands of FIFOs (and FIFO corresponding to each electrode), the information from the FIFOs can be multiplexed down to a single data stream so a single processor can read all the channels.

After the capture of these values is complete, the controller 500 can assert the RESET signal which discharges the peak-storage capacitor 455 of the peak detector circuit 460 (and, optionally, resets the value of the counter 480). It then de-asserts the PWR and ENAB signals, allowing the system to return to a low-power state and to await the next detected action potential.

As previously indicated, the data representing the detected action potential (e.g., values representing peak voltage and peak time) can be multiplexed with data from other electrodes (e.g., in an electrode array) over a serial bus to an electronics platform (e.g., electrics platform 140 of FIG. 1), inside the skull but not embedded in the brain. The multiplexed data from all the electrodes can be sent wirelessly to an external controller or other device outside of the skull (e.g. the external controller 160 of FIG. 1).

According to some embodiments, rather than utilizing the circuit 400 and controller 500, electrodes may implement similar functionality using a digital design.

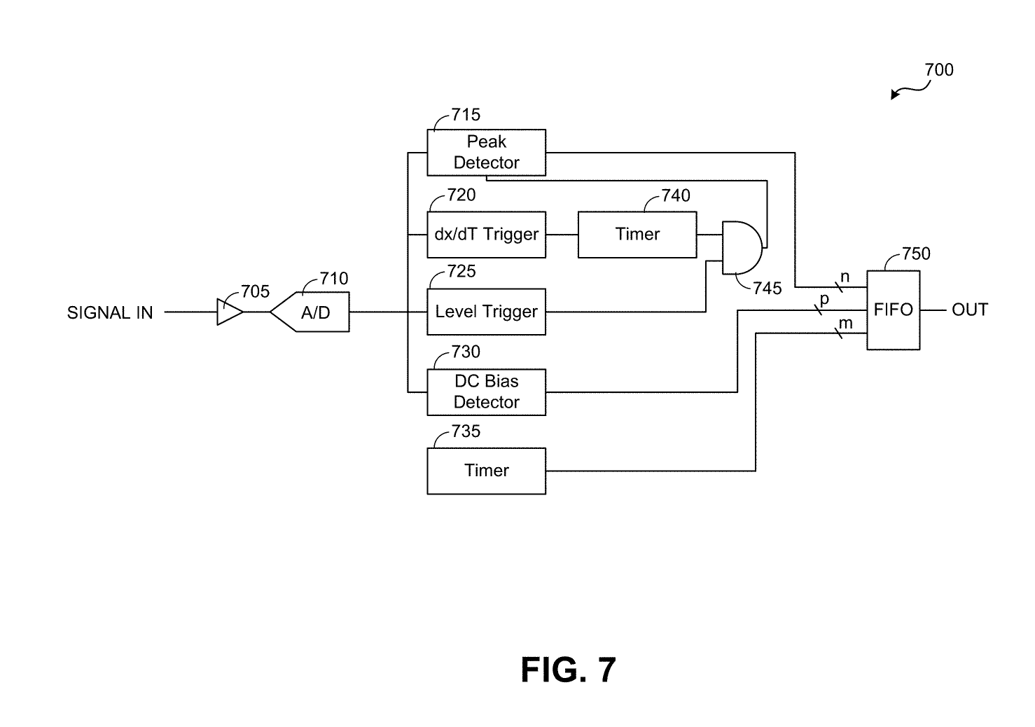

FIG. 7 is a block diagram of a digital circuit 700 used to detect and capture information regarding action potentials, according to an embodiment. Here, the SIGNAL IN signal is input to a low-power amplifier 705, and A/D converter 710 converts the analog signal to a digital bit stream. (It can be noted that, in some embodiments, a low-power amplifier 705 may not be needed, depending on the capabilities of the A/D converter 710.) Once in a digital bit stream, a processor or state machine can perform functions analogous to those illustrated in FIGS. 4-6 and described above to output values indicative of the peak signal, a time the action potential was detected, and (optionally) a local potential to a FIFO 750.

More specifically, a differentiator 720 can determine whether an incoming signal has a threshold slope and, if so, provide a true signal to a timer 740, which can output a true signal if the detected slope last for a threshold amount of time. If so, in the output a true signal to the first input of a AND gate 745. A level trigger 725 can determine whether the incoming signal has a threshold amplitude and, if so, output a true signal to the second input of the AND gate 745. Upon receiving true inputs, the AND gate 745 provides a true output to the peak detector 715, which captures a digital value representing a peak value of the amplitude of the incoming signal, and provides it to the FIFO 750. At this time, the FIFO 750 can also capture a value of the local potential from the DC bias detector 730, and the value of the time the action potential was detected from the timer 735. It can be noted that the block diagram of the digital circuit 700 is intended to show all the major functional blocks without some of the additional detail shown in FIGS. 4-5. A person of ordinary skill in the art will recognize, for example, that various additional signals may be communicated (e.g. in a manner similar to FIGS. 4-5) to cause the FIFO 750, timer 735, and/or DC bias detector 730 to store a value, etc.

The digital circuit 700 as noted above, the digital circuit 700 can be used as an alternative embodiment to the circuit 400 and controller 500 of FIGS. 4 and 5. This may have advantages when size is an issue, since they digital circuit 700 does not include physically large structures, such as capacitors. In alternative embodiments, the peak detector 715 may be replaced with a peak capture circuit, which provides the digital values of the input signal to the FIFO 750 to capture not only the peak value of the detected action potential, but the entire wave form, if desired.

Depending on desired functionality, some embodiments may utilize a hybrid approach, combining digital aspects of the digital circuit 700 with analog aspects of the circuit 400. For example, according to some embodiments, and analog comparator may be utilized in a manner similar to the circuit 400 to detect an action potential in the incoming signal (using voltage, slope, and/or timing thresholds), then activating digital circuitry to sample the waveform and/or peak amplitude of the action potential. In some embodiments, a length of time of the digital sampling may be predetermined (e.g., a predetermined period of time sufficient to sample any expected action potential). A sampling rate of the digital sampling may be configured to take a certain number of samples (e.g., 5-10 samples) during the predetermined period of time for digital sampling. In some embodiments, the sampling rate may be configurable and/or dynamic to enable higher sampling resolution when needed.

It will be understood that, for the circuit 400, digital circuit 700, and/or other embodiments, the resolution of the values of the timestamp, peak amplitude (and/or waveform), and local potential can vary, depending on desired functionality. An example sampling rate of the A/D converter converting the incoming peak or waveform of the action potential (e.g., A/D converter 470 or A/D converter 710) may be 10 kHz, although alternative embodiments may have a sampling rate higher or lower than this. In some embodiments, the number of bits of the timestamp (m) may be 16, and a number of bits for the amplitude maybe 24: 12 for the peak (n), and 12 for the local potential (p). Again, alternative embodiments may vary.

In some embodiments, rather than a peak or a waveform, amplitude of a detected action potential may be conveyed by integration (i.e., area under the curve of the action potential's amplitude). Such embodiments may, for example, replace a peak detector circuit 460 with an integration circuit, which can also involve charging a capacitor. In some embodiments, both peak detection and integration may be performed, and values for both the peak amplitude and the area under the curve may be transmitted.

As noted above, the voltage, slope, and/or time thresholds may be adjusted, based on desired functionality. In some embodiments, these thresholds may be programmable, and may be altered in the field after deployment (e.g., after electrodes are implanted). Adjusting these values to nonzero values helps ensure action potentials are captured efficiently, and noise and other undesirable signals are filtered out. These thresholds may be adjusted, for example, to filter out all signals in the graph 300 of FIG. 3 except for the high-voltage series 310. The more selective the filtration and fewer detected action potentials, the less bandwidth will be required to transmit information regarding the detected action potentials to a controller device. In some embodiments, each of these thresholds could be separately managed and/or driven by a pulse-duration modulation (PDM) or digital to analog (D/A) converter driven, for example, by a processor. In some embodiments, the external controller receiving the data could provide feedback to the electrodes to adjust these voltage thresholds. In some embodiments, these voltage thresholds may be user-defined (e.g., by a doctor or healthcare provider).

Once set, these voltage thresholds rarely need to be adjusted. That said, in many instances scar tissue will begin to develop around the electrodes, which can reduce detected voltages. In such instances, one or more of the thresholds may be lowered to adapt to this phenomenon. This phenomenon could be detected by determination that less activity is being reported by the system in cases where normal activity would ordinarily be expected.

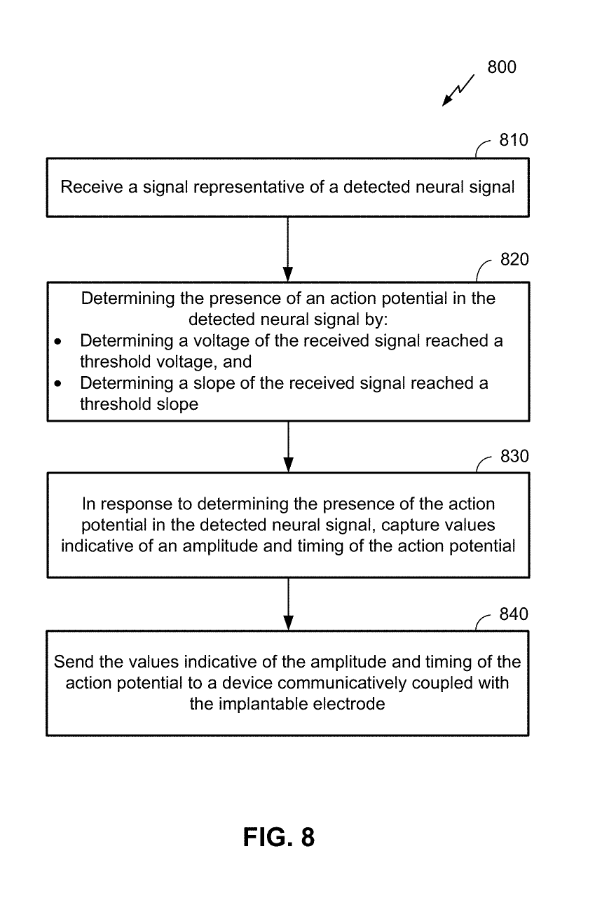

FIG. 8 is a flow diagram of an example method 800 of capturing neural signals with an electrode, according to an embodiment. The functionality of the circuits illustrated in FIGS. 4-7, for example, can be generally described as performing the method 800. But it will be understood that this functionality may be performed by alternative hardware and/or software of an electrode.

At block 810, a signal representative of a detected neural signal is received. Here, the received signal may comprise an output signal from a sensor (e.g., the SIGNAL IN signal described in the embodiments above), or signals derived therefrom (which may be buffered, amplified, digitized, etc.) Accordingly, means for performing the functionality at block 810 may comprise, for example, a low-power amplifier (e.g., the low-power amplifier 405 of FIG. 4 or the low-power amplifier 705 of FIG. 7).

At block 820, the presence of an action potential in the detected neural signal is determining by determining a voltage of the received signal reached a threshold voltage and determining a slope of the received signal reached a threshold slope. As described in the embodiments above, different circuits may be utilized to separately determine whether these different thresholds have been met. Moreover, the threshold voltage and/or threshold slope may be dynamically configurable, depending on desired functionality. Means for performing the functionality at block 820 may comprise, for example, DC bias detector 410, comparators 415 and 435, differentiator 425, AND gate 430 of FIG. 4, and controller 500 of FIG. 5, or differentiator 720 and level trigger 725 of FIG. 7. Moreover, as previously indicated, the threshold voltage and/or threshold slope may be established by voltage set by PDM or D/A converter, which may be driven by a processor.

At block 830, the functionality comprises, in response to determining the presence of the action potential in the detected neural signal, capturing values indicative of an amplitude and timing of the action potential. As illustrated above, circuits for performing such functionality may be inactive until an action potential is detected, e.g., for power conservation and/or heat dissipation reasons. Moreover, as noted above, the values indicative of an amplitude may comprise a peak value, waveform, and/or integration value (area under the curve of the action potential). Means for performing the functionality at block 830 may comprise, for example, high-power amplifier 450, switch 465, peak detector circuit 460, A/D converter 470, counter for a zero, FIFO 475, and controller 500 of FIG. 5, or peak detector 715, timer 735, and FIFO 750 of FIG. 7.

At block 840, the values indicative of the amplitude and timing of the action potential are sent to a device communicatively coupled with the electrode. As noted above, this may comprise communicating information to an electronics platform (e.g., electronics platform 140 of FIG. 1) and/or to a device outside the body (e.g., external controller 160 of FIG. 1). As noted above, such communication can be sent via wireless or wired means, which may occur after multiplexing information from a plurality of electrodes. Means for performing the functionality at block 830 may comprise a transmitter or transceiver configured to communicate with the device via electrical, optical, RF, and/or other means.

Alternative embodiments of the method 800 may include additional or alternative functionality. For instance, the method may include adjusting the threshold voltage, the threshold slope, or both. In some embodiments, the electrode may be wholly or partially implantable (e.g., wholly or partially subcutaneous). In some embodiments, the electrode may be able to detect neural activity without implantation (e.g., externally). In some embodiments, determining the presence of an action potential in the detected neural signal may further comprise determining whether the received signal has met or exceeded a threshold slope for at least a threshold amount time. In some embodiments, this threshold amount of time may be adjustable (e.g., using a programmable timer). In some embodiments, capturing the values indicative of the amplitude and the timing of the action potential is performed by a peak detector coupled with an analog-to-digital converter and/or a counter. In some embodiments, the counter may be configured to be reset in response to the detection of the action potential. Additionally, or alternatively, a local potential of the detected neural signal may be detected, and the method further includes sending a value indicative of an amplitude of the detected local potential.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein.

Therefore, it is intended that claimed subject matter not be limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

What is claimed is:

1. An electrode of a neural recording system, the electrode comprising:
a neural sensor configured to provide an output signal representative of a detected neural signal;
detection circuitry coupled to the neural sensor and configured to determine, from the output signal of the neural sensor, the presence of an action potential in the detected neural signal, the detection circuitry comprising:
voltage threshold circuitry configured to detect when a voltage of the output signal of the neural sensor reaches a threshold voltage, and
slope-detection circuitry configured to detect when a rising slope of the output signal of the neural sensor reaches a threshold slope;
capture circuitry coupled to the detection circuitry and configured to, based on the determined presence of an action potential, capture a value indicative of an amplitude of the action potential and a value indicative of timing of the action potential; and
transmission circuitry coupled to the capture circuitry and configured to send the value indicative of the amplitude of the action potential and the value indicative of the timing of the action potential to a device communicatively coupled with the electrode.

2. The electrode of claim 1 further comprising circuitry configured to enable adjustment of the threshold voltage, the threshold slope, or both.

3. The electrode of claim 1 wherein the slope-detection circuitry further comprises time threshold circuitry configured to provide an output based on whether the output signal of the neural sensor has met or exceeded the threshold slope for at least a threshold amount of time.

4. The electrode of claim 3 further comprising circuitry configured to enable adjustment of the threshold amount of time.

5. The electrode of claim 1, wherein the capture circuitry comprises a peak detector coupled with an analog-to-digital converter.

6. The electrode of claim 1, further comprising a counter configured to provide the value indicative of the timing of the action potential.

7. The electrode of claim 6, wherein the counter is configured to be reset in response to the detection of the action potential.

8. The electrode of claim 1, further comprising local potential detection circuitry configured to detect, from the output signal of the neural sensor, a local potential of the detected neural signal, wherein the transmission circuitry is further coupled to the local potential detection circuitry and configured to send a value indicative of an amplitude of the detected local potential to the device communicatively coupled with the electrode.

9. A method of operating an electrode of a neural recording system, the method comprising:
receiving, from a neural sensor, an output signal representative of a detected neural signal;
determining, from the output signal of the neural sensor, the presence of an action potential in the detected neural signal, by:
determining a voltage of the output signal of the neural sensor reached a threshold voltage, and
determining a rising slope of the output signal of the neural sensor reached a threshold slope;
in response to the determination of presence of the action potential, capturing a value indicative of an amplitude of the action potential and a value indicative of timing of the action potential; and
sending the value indicative of the amplitude of the action potential and the value indicative of the timing of the action potential to a device communicatively coupled with the electrode.

10. The method of claim 9 further comprising adjusting the threshold voltage, the threshold slope, or both.

11. The method of claim 9 further comprising providing an output based on whether the output signal of the neural sensor has met or exceeded the threshold slope for at least a threshold amount of time.

12. The method of claim 11 further comprising adjusting the threshold amount of time.

13. The method of claim 9, further comprising operating a counter to provide the value indicative of the timing of the action potential.

14. The method of claim 13, further comprising resetting the counter in response to the detection of the action potential.

15. The method of claim 9, further comprising:
detecting, from the output signal of the neural sensor, a local potential of the detected neural signal; and
sending a value indicative of an amplitude of the detected local potential to the device communicatively coupled with the electrode.

16. An apparatus comprising:
means for receiving, from a neural-sensing means, an output signal representative of a detected neural signal;
means for determining, from the output signal of the neural-sensing means, the presence of an action potential in the detected neural signal, by:
determining a voltage of the output signal of the neural-sensing means reached a threshold voltage, and
determining a rising slope of the output signal of the neural-sensing means reached a threshold slope;
means for capturing a value indicative of an amplitude of the action potential and a value indicative of timing of the action potential; and
means for sending the value indicative of the amplitude of the action potential and the value indicative of the timing of the action potential to a device communicatively coupled with the apparatus.

17. The apparatus of claim 16, further comprising means for adjusting the threshold voltage, the threshold slope, or both.

18. The apparatus of claim 16, further comprising means for providing an output based on whether the output signal of the neural-sensing means has met or exceeded the threshold slope for at least a threshold amount of time.

19. The apparatus of claim 18, further comprising means for adjusting the threshold amount of time.

20. The apparatus of claim 16, further comprising means for operating a counter to provide the value indicative of the timing of the action potential.

21. The apparatus of claim 20, further comprising means for resetting the counter in response to the detection of the action potential.

22. The apparatus of claim 16, further comprising:
means for detecting, from the output signal of the neural-sensing means, a local potential of the detected neural signal; and
means for sending a value indicative of an amplitude of the detected local potential to the device communicatively coupled with the apparatus.

23. A non-transitory computer-readable medium having instructions embedded thereon for operating an electrode of a neural recording system, the instructions comprising computer code for:
receiving, from a neural sensor, an output signal representative of a detected neural signal;
determining, from the output signal of the neural sensor, the presence of an action potential in the detected neural signal, by:
determining a voltage of the output signal of the neural sensor reached a threshold voltage, and
determining a rising slope of the output signal of the neural sensor reached a threshold slope;
capturing a value indicative of an amplitude of the action potential and a value indicative of timing of the action potential; and
sending the value indicative of the amplitude of the action potential and the value indicative of the timing of the action potential to a device communicatively coupled with a computing device operating the electrode.

24. The non-transitory computer-readable medium of claim 23, wherein the instructions further comprise computer code for adjusting the threshold voltage, the threshold slope, or both.

25. The non-transitory computer-readable medium of claim 23, wherein the instructions further comprise computer code for providing an output based on whether the output signal of the neural sensor has met or exceeded the threshold slope for at least a threshold amount of time.

26. The non-transitory computer-readable medium of claim 25, wherein the instructions further comprise computer code for adjusting the threshold amount of time.

27. The non-transitory computer-readable medium of claim 23, wherein the instructions further comprise computer code for operating a counter to provide the value indicative of the timing of the action potential.

28. The non-transitory computer-readable medium of claim 27, wherein the instructions further comprise computer code for resetting the counter in response to the detection of the action potential.

29. The non-transitory computer-readable medium of claim 23, wherein the instructions further comprise computer code for:
detecting, from the output signal of the neural sensor, a local potential of the detected neural signal; and
sending a value indicative of an amplitude of the detected local potential to the device communicatively coupled with the electrode.
